(19) United States
(12) Patent Application Publication
(10) Pub. No.: US 2022/0157439 A1
(43) Pub. Date: May 19, 2022

(54) Interaction Method, Electronic Device, and Storage Medium

(71) Applicant: BOE Technology Group Co., Ltd., Beijing (CN)

(72) Inventor: Lei ZHAO, Beijing (CN)

(21) Appl. No.: 17/355,496

(22) Filed: Jun. 23, 2021

(30) Foreign Application Priority Data
Nov. 18, 2020 (CN) 202011294059.8

(51) Int. Cl. G16H 40/20; H04B 5/00; G06F 3/14

(52) U.S. Cl. CPC G16H 40/20; G06F 3/14; H04B 5/0031

(57) ABSTRACT

The embodiments of the present disclosure disclose an interaction method, an electronic device and a storage medium. In one implementation, the method is applied to an electronic device with an interaction device and comprises: receiving a shift-handover record, and displaying a first interface, wherein the first interface displays shift-handover information corresponding to the shift-handover record, and a shift-takeover confirm control, and the shift-handover record is a shift-handover record by medical staff; receiving shift-takeover permission application information and verifying a shift-takeover permission; and confirming that a shift exchange is completed in response to an operation on the shift-takeover confirm control.

### CROSS REFERENCE OF RELEVANT APPLICATION

The present disclosure claims the priority of the Chinese patent application filed on Nov. 18, 2020 before the Chinese Patent Office with the present disclosure number of 202011294059.8 and the title of “INTERACTION METHOD, ELECTRONIC DEVICE, AND STORAGE MEDIUM”, which is incorporated herein in its entirety by reference.

### TECHNICAL FIELD

The present disclosure relates to the technical field of computers, in particular to an interaction method, an electronic device and a storage medium.

### BACKGROUND

At present, doctors and nurses work in turns in the hospital, particularly in the inpatient department. Generally, several groups of medical staff are response for one ward in turns. At present, the medical staff exchanges shifts typically as follows: medical staff to be relieved manually fills in a paper shift-handover record with ward information, patent information, medical supply information of patents undertaken and then hands the paper shift-handover record to medical staff who is about to take his/her turn on duty, the medical staff to be on duty signs on the paper shift-handover record for confirmation to generate a shift-exchange report to be achieved, and thus, a shift exchange of the medical staff is completed.

### SUMMARY

The objective of the present disclosure is to provide an interaction method, an electronic device and a storage medium.

The present disclosure adopts the following technical solutions:

In a first aspect, the present disclosure provides an interaction method, which is applied to an electronic device with an interaction device and comprises:

receiving a shift-handover record, and displaying a first interface, wherein the first interface displays shift-handover information corresponding to the shift-handover record, and a shift-takeover confirm control, and the shift-handover record is a shift-handover record by medical staff;

receiving shift-takeover permission application information and verifying a shift-takeover permission; and

confirming that a shift exchange is completed in response to an operation on the shift-takeover confirm control.

Optionally, before the step of receiving the shift-handover record, the method further comprises:

displaying a second interface, wherein the second interface displays a shift-handover record create control;

receiving a shift-handover permission application information and verifying a shift-handover permission; and

acquiring shift-handover information and generating the shift-handover record in response to an operation on the shift-handover record create control.

Optionally, the step of acquiring the shift-handover information comprises:

acquiring the shift-handover information from a database; and/or

displaying a third interface that display an input control, and acquiring the shift-handover information input by means of the input control.

Optionally, the step of the receiving shift-handover permission application information and verifying the shift-handover permission comprises: receiving shift-handover permission application information in NFC data and verifying the shift-handover permission.

The step of receiving the shift-takeover permission application information and verifying the shift-takeover permission: receiving shift-takeover permission application information in NFC data and verifying the shift-takeover permission.

Optionally, the shift-turnover information corresponding to the shift-turnover record includes general ward information and patient information corresponding to a current shift exchange, and the patient information includes basic information, illness condition and nursing information of patents.

Optionally, the shift-handover information corresponding to the shift-handover records further includes: general ward information in a previous shift exchange, and patient illness information, corresponding to the previous shift exchange, of patients whose patient illness information corresponding to the current shift exchange has changed with respect to the previous shift exchange.

Optionally, the second interface also displays a shift-handover record format set control, and the method further comprises:

receiving a first set permission application information and verifying a first set permission; and

setting a format of the shift-turnover record in response to an operation on the shift-handover record format set control.

Optionally, the second interface also displays patient controls corresponding to patients in wards, and the method further comprises:

displaying a fourth interface in response to an operation on the patient controls, wherein the fourth interface displays corresponding patient information, and the patient information includes basic information, illness information and nursing information of the patients.

Optionally, the second interface also displays a display set control, and the method further comprises:

receiving second set permission application information and verifying a second set permission; and

setting information displayed by the second interface, and contents and display parameters of the controls in response to an operation on the display set control.

Optionally, after the step of confirming that the shift exchange is completed, the method further comprises: displaying the second interface.

In a second aspect, the present disclosure provides an electronic device, comprising: a processor, a memory and an interaction device, wherein the memory and the interaction device are coupled to the processor, the memory is used to store a computer program code which includes a computer instruction, and when the processor reads the computer instruction from the memory, the electronic device is enabled to implement the interaction method provided in the first aspect of the present disclosure.

Optionally, the electronic device is an electronic whiteboard device installed in a medical office area.

In a third aspect, the present disclosure provides a computer storage medium, comprising a computer instruction which enables, when run on a terminal, the terminal to implement the interaction method provided in the first aspect of the present disclosure.

In a fourth aspect, the present disclosure provides an electronic device, wherein the electronic device comprises:

a memory configured for storing a computer instruction executable by the processor; and

one or more processors, wherein the processors are configured for executing the computer instruction, to implement the interaction method according to claim 1.

Optionally, before the operation of receiving the shift-handover record, the processors further execute the operations comprising:

displaying a second interface, wherein the second interface displays a shift-handover record create control;

receiving a shift-handover permission application information and verifying a shift-handover permission; and

acquiring shift-handover information and generating the shift-handover record in response to an operation on the shift-handover record create control.

Optionally, the operation of acquiring the shift-handover information comprises at least one of:

acquiring the shift-handover information from a database; and

displaying a third interface that display an input control, and acquiring the shift-handover information input by means of the input control.

Optionally,

the operation of the receiving shift-handover permission application information and verifying the shift-handover permission comprises:

receiving shift-handover permission application information in NFC data and verifying the shift-handover permission.

The above description is merely a summary of the technical solutions of the present disclosure. In order to more clearly know the elements of the present disclosure to enable the implementation according to the contents of the description, and in order to make the above and other purposes, features and advantages of the present disclosure more apparent and understandable, the particular embodiments of the present disclosure are provided below.

### BRIEF DESCRIPTION OF THE DRAWINGS

For clearer descriptions of the technical solutions in the embodiments of the present disclosure, the following briefly introduces the accompanying drawings required for describing the embodiments of the present disclosure. Apparently, the accompanying drawings in the following description show merely some embodiments of the present disclosure, and a person of ordinary skill in the art may still derive other drawings from these accompanying drawings without creative efforts.

The specific implementations of the present disclosure will be described in further detail below in conjunction with accompanying drawings.

FIG. 1 illustrates a first structural diagram of an electronic device provided by one embodiment of the present disclosure.

FIG. 2 illustrates a second structural diagram of the electronic device provided by one embodiment of the present disclosure.

FIG. 3 illustrates a flow diagram of an interaction method provided by one embodiment of the present disclosure.

FIG. 4 illustrates a schematic diagram of a user interface.

FIG. 5 illustrates a schematic diagram of another user interface.

FIG. 6 illustrates a schematic diagram of another user interface.

FIG. 7 illustrates a schematic diagram of another user interface.

FIG. 8 illustrates a schematic diagram of another user interface.

FIG. 9 is a third structural diagram of the electronic device according to one embodiment of the present disclosure.

### DETAILED DESCRIPTION

To more clearly explain the present disclosure, the present disclosure will be further described below in conjunction with the embodiments and accompanying drawings. In the drawings, similar components are represented by identical reference signs. Those skilled in the art would appreciate that the contents specifically described below are illustrative instead of being restrictive, and should not be construed as limiting the protection scope of the present disclosure.

In the description of the embodiments of the present disclosure, “I” refers to “or”, unless otherwise specified. For example, AB may refer to A or B. In this specification, “and/or” merely describes an association of associated objects, and may indicate there associations. For example, A and/or B may indicate that only A exists, both A and B exist, or only B exists.

Terms “first” and “second” in the following description are merely for a descriptive purpose, and should not be construed as indicating or implying relative importance or implicitly indicating the number of technical features referred to. Thus, a feature defined by “first” or “second” may explicitly or implicitly refer to the inclusion of one or more said features. In the description of the embodiments of the present disclosure, “multiple” refers to two or more, unless otherwise specified.

In the embodiments of the present disclosure, the term such as “illustrative” or “for example” is used as to expressing an example, illustration or description. Any embodiment or design solution defied by “illustrative” or “for example” of the present disclosure should not be construed as being more preferable or advantageous than other embodiments or design solutions. To be specific, the term such as “illustrative” or “for example” is used to presenting relevant concepts in a specific manner.

Illustratively, the electronic device in the present disclosure is a large-screen electronic whiteboard device with a touch screen over 55 inches, is installed at a nurse station in a surgical ward of the inpatient department of a hospital, and is used by nurses to check the condition of wards and the condition of patents in the wards and to exchange shifts.

FIG. 1 illustrates a structural diagram of an electronic device 100.

The electronic device 100 comprises a memory 110 and a processor 120. It should be understood that components of the electronic device 100 shown in FIG. 1 are illustrative, and are not restrictive, and the electronic device 100 may also comprise other components according to actual application requirements. For example, the electronic device 100 may adopt a Windows operating system, an Android operating system, or the like.

For example, the memory 110 is used for storing a computer readable instruction in a non-transitory manner, and the processor 120 is used for running the computer readable instruction.

For example, as shown in FIG. 1, the electronic device 100 may further comprise a touch device 130. The touch device 130 is configured to acquire a touch operation applied to a work surface of the touch device 130. For example, the touch device 130 may be any input devices with a touch function such as an electronic pen, a touch screen, a mouse, a touchpad and an interactive whiteboard. For example, the touch screen may be a capacitive touch screen such as a self-capacitive touch screen or a mutually-capacitive touch screen, a resistive touch screen, an SAW touch screen, an infrared touch screen, or the like.

For example, the touch device 130 may comprise a touch sensor and a controller (such as a drive IC), wherein the controller receives an electric signal collected by the touch screen, processes the electric signal to obtain a touch signal, and supplies the touch signal to the processor for further processing. The embodiments of the present disclosure have no limitation to the type, structure and communication manner of the touch device 130. The touch device 130 has a work surface with a certain area, users may write on the work surface of the touch device 130 directly with a finger or may write on the work surface of the touch device 130 with an active touch pen or a passive touch pen, and the embodiments of the present disclosure has no limitation in this aspect. Here, the work surface refers to a surface for detecting the touch operation performed by users, such as a touch surface of the touch device 130.

For example, as shown in FIG. 1, the electronic device 100 may further comprise a display device 140. The display device 140 is configured to display interactive interfaces. For example, the display device 140 may comprise a display screen, a projector, or the like. For example, the display screen of the display device 140 may be an LCD display screen, an OLCD display screen, a QLED display screen, a projection component, a VR head-mounted display device (such as a VR helmet or VR glasses), an AR display device, or the like, and the embodiments of the present disclosure have no limitation in this aspect.

For example, in some embodiments, the touch device 130 and the display device 140 may be integrated into, such as, a touch display screen that has both a touch function and a display function.

For example, the memory 110 and the processor 120 may be integrated in the touch display screen; or, the memory 110 and the processor 120 may be integrated in a cloud server.

For example, the electronic device 100 may further comprise a communication module used for communication between the electronic device 100 and other electronic devices. For example, when the electronic device 100 is applied to a nurse station, the communication module may transmit interactive interfaces to computer devices installed in doctor's offices, and the computer devices in the doctor's offices may display and store the interactive interfaces, so that doctors may check the conditions of wards and patients in their offices.

For example, the processor 120 may control other components in the display device 100 to fulfill desired functions. The processor 120 may be a device capable of processing data and/or executing a program such as a central processing unit (CPU) or a tensor processing unit (TPU). The central processing unit (CPU) may adopt an X86 or ARM architecture.

For example, the memory 110 may comprise any combination of one or more computer program products, and the computer program products may comprise computer readable storage media in different forms, such as a volatile memory and/or a nonvolatile memory. For example, the volatile memory may be a random access memory (RAM) and/or a cache memory (cache). For example, the nonvolatile memory may be a read only memory (ROM), a hard disk, an eclectically programmable read only memory (EPROM), a portable CD read only memory (CD-ROM), a USB memory, a flash memory, or the like. One or more computer readable instructions may be stored in the computer readable storage medium and may be run by the processor 120 to fulfill different functions of the electronic device 100.

For example, the memory 110, the processor 120, the touch device 130 and the display device may communicate with one another through a network. The network may be a wireless network, a wired network, and/or any combination of the wireless network and the wired network. Or, the network may be a local area network, an internet, a telecommunication network, an Internet of Things based on the internet and/or the telecommunication network, and/or any combination of these networks. For example, the wired network may carry out communication by means of a twisted pair, a coaxial cable, an optical fiber transmission, or the like. For example, the wireless network may carry out communication by means of a 3G/4G/5G mobile communication network, Bluetooth, Zigbee, Wifi, or the like. The present disclosure has no limitation to the type and function of the network.

A software system of the electronic device 100 may adopt a layered architecture, an event-driven architecture, a micro-kernel architecture, a micro-service architecture, or a cloud architecture. In the embodiments of the present disclosure, the software structure of the electronic device 100 is illustratively described with an Android system of a layered architecture as an example.

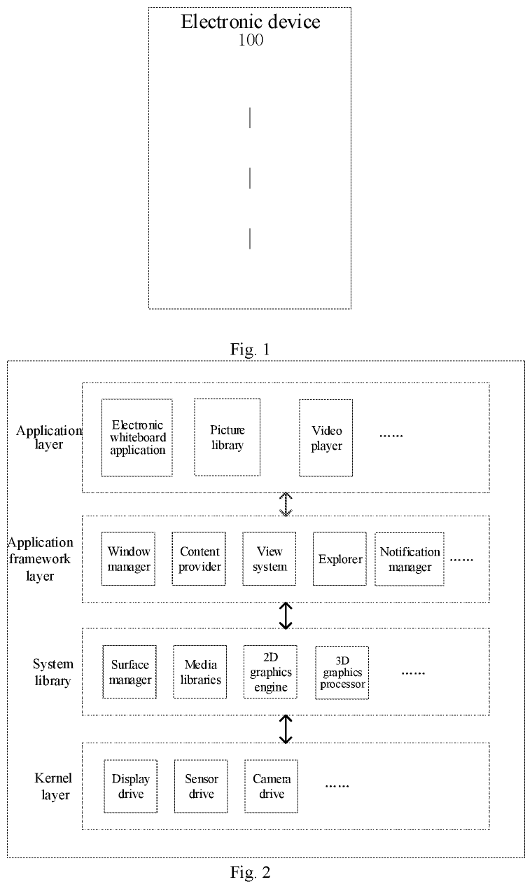

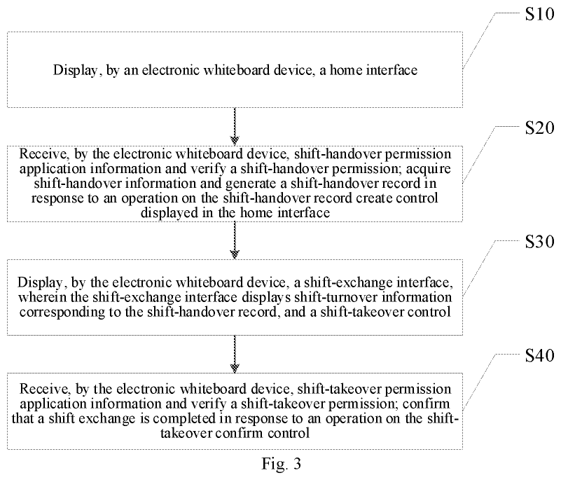

FIG. 2 illustrates a block diagram of the software structure of the electronic device 100 according to one embodiment of the present disclosure.

Software is divided by the layered architecture into a plurality of layers, and each layer has an explicit function. All the layers communicate with one another by means of software interfaces. In some embodiments, the Android system is divided into four layers, which are respectively an application layer, an application framework layer, an Android runtime and system library, and a kernel layer.

The present disclosure layer may comprise a series of application packages. As shown in FIG. 2, the present disclosure packages may comprise an electronic whiteboard application, a picture library, a video player, and other applications such as a camera, a calendar, a communication application, a map, a navigator, WLAN, Bluetooth and music.

In some embodiments of the present disclosure, the electronic whiteboard application may provide interfaces for interaction between the electronic device 100 and users (for example, nurses).

The present disclosure framework layer provides an application programming interface (API) and a programming framework for the present disclosures in the present disclosure layer, and comprises some pre-defined functions.

As shown in FIG. 2, the present disclosure framework layer may comprise a window manger, a content provider, a view system, an explorer, a notification manager, and the like.

The window manager is used for managing window programs, and is able to acquire the size of the display screen and determine whether there is a status bar, whether the screen is locked, and whether there is a screenshot.

In some embodiments of the present disclosure, the window manager may specifically be a window manager service (WMS), in which information of application windows displayed in the current screen is stored, such as the number of the present disclosure windows displayed in the current screen.

The content provider is used for storing and acquiring data, and allowing the data to be accessed by applications. The data may comprise videos, images, audio data, files, and the like.

The view system comprises visible controls, such as a control for displaying characters and a control for displaying pictures, and may be used for establishing an application. The display interface may be composed of one or more views. For example, a display interface with a massage notification icon may comprise a view for displaying characters and a view for displaying pictures.

The explorer provides various resources for the present disclosures, such as localizable strings, icons, pictures, layout files, video files, and the like.

The notification manager enables the present disclosures to display notifications in the status bar, is used to convey messages for informing users of notifications, may automatically disappear after a short stay, and does not need to interact with users. For example, the notification manager is used for informing users of the completion of download, message notifications, and the like. The notification manager may provide a message appearing in the status bar at the top of the system in the form of a chart or scrollbar text such as a notification of an application running background, or a notification appearing on the screen in the form of a dialog box such as a prompt text information in the status bar, prompt sounds, and flashing of an indicator light.

Android Runtime comprises a core library and a virtual machine, and is responsible for scheduling and managing the Android system.

The core library comprises two parts, wherein one part is a functional function to be called by a java language, and the other part of the core library for the Android system.

The present disclosure layer and the present disclosure framework layer run in the virtual machine. The virtual machine executes java files in the present disclosure layer and the present disclosure framework layer into binary files, and is used for management of the life cycle of an executed object, stack management, thread management, security and exception management, junk collection, and the like.

The system library may comprise multiple functional modules, such as a surface manager, media libraries, a 2D graphics engine (such as SGL), and a 3D graphics processing library (such as OpenGL ES).

The surface manager is used for managing display sub-systems and merging 2D and 3D graphics for multiple applications.

The media libraries support multiple common audio files, playback and recording of videos, static image files, and the like, and may support many video and audio coding formats, such as MPEG4, H.264, MP3, AAC, AMR, JPG and PNG.

The 3D graphics processing library is used for realizing 3D graphics drawing, image rendering and synthesis, layer processing, and the like.

The 2D graphics engine is a drawing engine for drawing 2D graphics.

The kernel layer is located between hardware and software and comprises a display drive, a camera drive, an audio drive, a sensor drive, and the like.

The technical solutions involved in the following embodiments may be implemented by means of the electronic device 100 adopting the above-mentioned hardware architecture and software architecture.

According to an interaction method provided by the present disclosure, nurses may trigger the electronic device 100 by a preset operation for enabling a shift-exchange mode to turn a display home interface to a display shift-exchange interface, and then perform a pre-defined operation by means of a control displayed on the shift-exchange interface to complete shift-handover and shift-takeover to complete a shift-exchange task.

In some embodiments, the shift-exchange interface is, for example, a first interface, and the home interface is, for example, a second interface.

Below, the technical solutions provided by the embodiments of the present disclosure will be described in detail in conjunction with the accompanying drawings using an electronic whiteboard device as the electronic device 100, the electronic whiteboard device may have a touch screen over 55 inches.

As shown in FIG. 3, one embodiment of the present disclosure provides an interaction method which is applied to the electronic device such as the electronic whiteboard device with a touch screen over 55 inches, and the electronic whiteboard device is installed at a nurse station in a surgical ward. The interaction method is applied to the electronic whiteboard device to realize shift exchanges for nurses and comprises the following steps:

S10: displaying, by the electronic whiteboard device, a home interface, wherein the home interface displays ward information and patient information.

In some feasible implementations, the home interface displays patient controls corresponding to patients in wards, and the interaction method provided by this embodiment further comprises:

Displaying a patient detail interface in response to an operation on the patient controls, wherein the patient detail interface displays corresponding patient information, and the patient information includes basic information, illness information and nursing information of the patients.

The patient detail interface may be a fourth interface.

In this way, the electronic whiteboard device is able to display patient information on the home interface in the form of, such as, a list or a bed distribution graph, so nurses on duty may check the condition of the patients at any time and may perform an operation on the control corresponding to a certain patient, such as by clicking the control, to check detailed information of the patient.

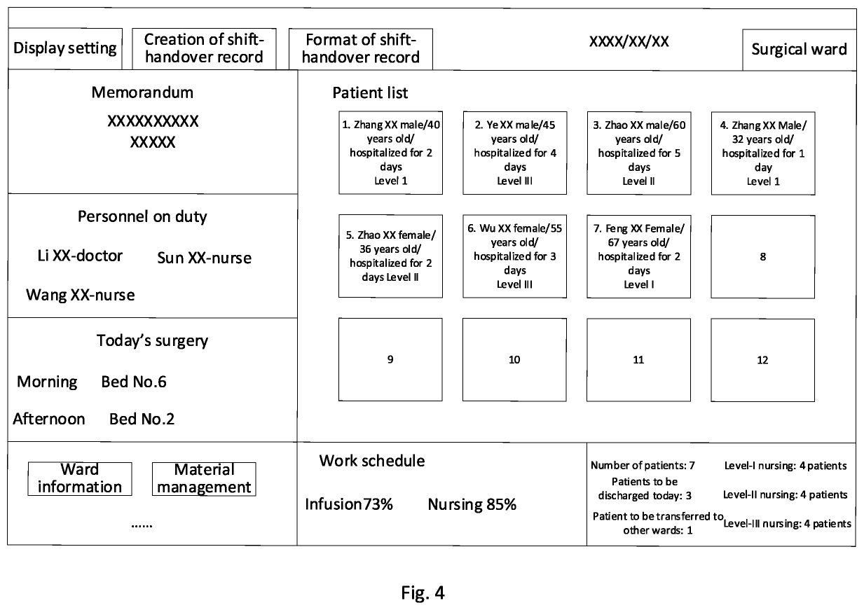

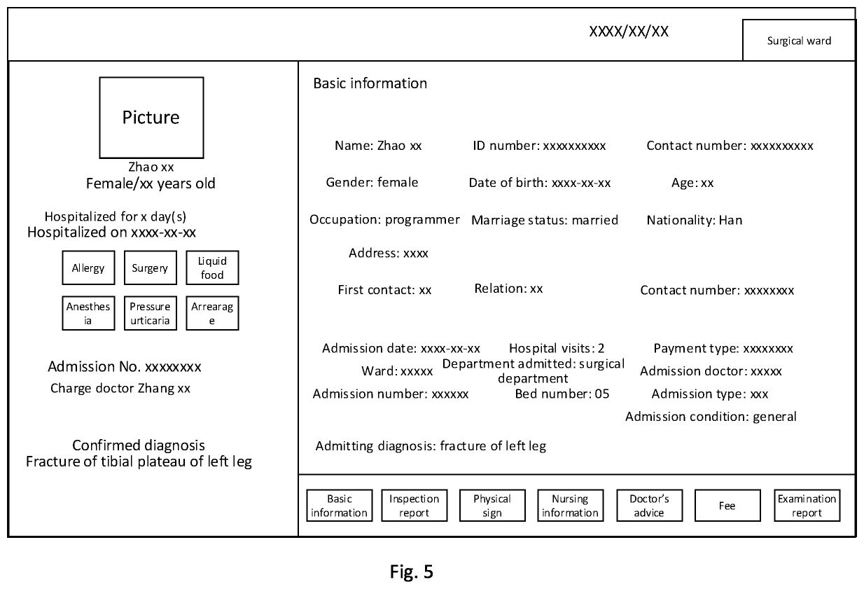

In one specific example, the home interface is shown in FIG. 4, the patient controls may be iconic controls, that are ordered according to bed numbers, in the area of “patient list”, and each iconic control shows the bed number, name, gender, age, admission time and nursing level of one patient; when a nurse performs a touch operation on a certain iconic control, such as, by clicking the iconic control with a finger, the electronic whiteboard device displays the patient detail interface of the corresponding patient, for example, when the nurse performs a touch operation on the iconic control corresponding to bed No. 5 by clicking the iconic control with a finger, the electronic whiteboard device displays the patient detail interface shown in FIG. 5, and the patient detail interface displays the basic information, illness information and nursing information of a patient Zhao XX on bed No. 5, such as the phone number, occupation and admitting diagnosis information shown in FIG. 5; wherein, other controls such as an inspection report control, a physical sign control and a nursing information control shown in the lower right of FIG. 5 may also be set in the patient detail interface, and when the nurse performs a touch operation on these controls, such as, by clicking the iconic controls with a finger, corresponding inspection report details, physical sign details and nursing details will be displayed by an interface jump or a pop-up window, so that all detailed information of the patient is displayed in an appropriate manner within a limited display area. In addition, the detailed information of different patients may be automatically displayed in turns by means of different detailed information display windows, and this embodiment has no limitation in this aspect.

It should be noted that the iconic controls, ordered according to the bed numbers, in the home interface shown in FIG. 4 are able to visually show beds occupied by patients and beds in a vacant state in the wards. For example, as can be seen from FIG. 4, beds No. 1-7 have been occupied by patients, and beds No. 8-12 are in a vacant state.

In some feasible implementations, the home interface also displays a display set control, and the interaction method provided by this embodiment further comprises:

receiving display set permission application information and verifying a display set permission; and

setting information displayed in the home interface and contents and display parameters of the control in response to an operation on the display set control.

The display set permission application information may be a second set permission application information.

In this way, personnel possessing a shift-handover record format set permission such as a charge nurse may modify the display contents and display parameters conveniently by means of the electronic whiteboard device, and thus, the interaction method provided by this embodiment is able to meet different requirements of different wards.

In some feasible implementations,

The step of receiving the display set permission application information and verifying the permission comprises: receiving the display set permission application information in NFC data and verifying the display set permission.

In this way, personnel with a display set permission such as a charge nurse may conveniently log in the electronic whiteboard device and verify their permission by means of a work card with an RFID or NFC tag. It may be understood that, in this implementation, the electronic whiteboard device should be provided with an NFC module such as an RFID reader or an NFC reader.

In addition to the above implementation in which the display set permission is verified by receiving the display set permission application information in NFC data, in this embodiment, the display set permission may also be verified in other verification manners such as by displaying a login control and receiving account information including a username and a password (the display set permission application information is the username and the password in this case) input by the login control, or by face recognition (the display set permission application information is a face image in this case), fingerprint recognition, iris recognition or voice recognition. It may be understood that when the face recognition, fingerprint recognition, iris recognition or voice recognition is correspondingly adopted for permission verification, the electronic whiteboard device should be provided with a camera, a fingerprint reader, an iris reader, a voiceprint reader, or a voice module.

In one specific example, the home interface is shown in FIG. 4, and the display set control is “display set” in FIG. 4; after the permission of a charge nurse is verified as valid, the charge nurse performs a touch operation on the display set control, such as, by clicking the display set control with a finger, the electronic whiteboard device displays “display set selection/input control” by an interface jump or a pop-up window in response to the touch operation, and modifies display contents or display parameters in response to an operation performed by the charge nurse on “display set selection/input control”. For example, the display contents may be modified by replacing “today's surgery” shown in FIG. 4 with infusion monitoring, and the display parameters may be modified by changing the location or size of the display areas, the font or color of characters, the type or color of the controls, or the like.

In one specific implementation, the home interface may also display other display contents such as infusion monitoring information, patient sign monitoring information, ward name, current time, memorandum, information of a nurse on duty, today surgery information in the wards, overall nursing schedule in the wards, general information of the wards, ward information controls and material management controls, and only part of the display contents are shown in FIG. 4. Wherein, these display contents may be played in turn or may be switched in response to a touch operation performed by sliding with a finger, and detailed information may be displayed in response to a touch operation on the controls.

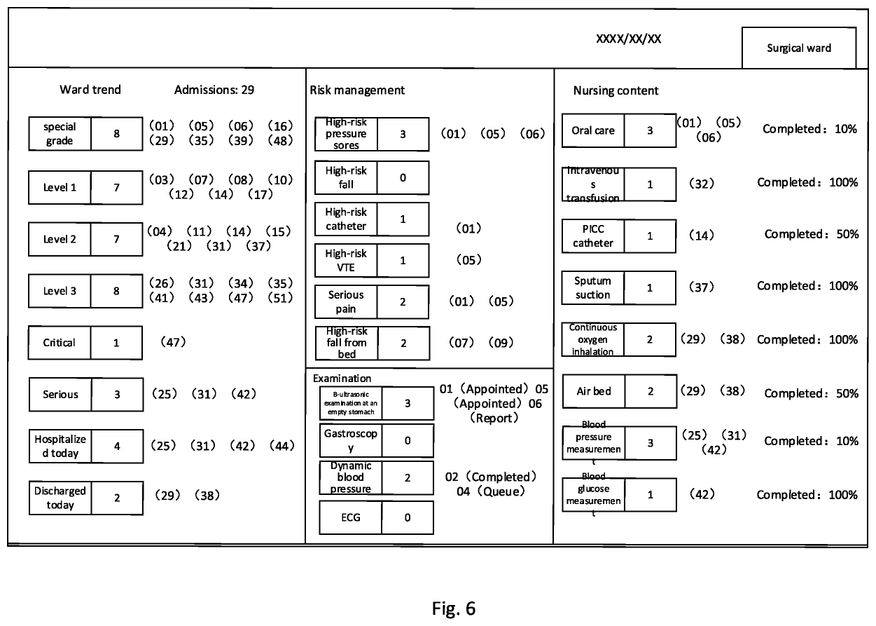

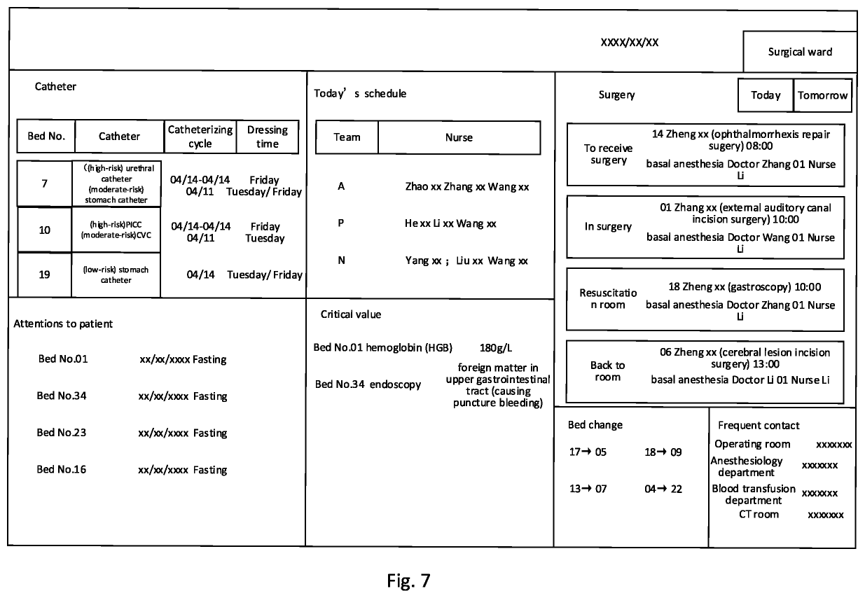

For example, when the nurse performs a touch operation on the “ward information” control in the home interface shown in FIG. 4, such as, by clicking the control with a finger, the electronic whiteboard device displays a ward detail interface, shown in FIG. 6, in response to the touch operation. When the nurse performs a touch operation on the ward detail interface shown in FIG. 6, such as, by leftward or rightward sliding with a finger, the electronic whiteboard device displays a ward detail interface, shown in FIG. 7, in response to the touch operation. Wherein, ward details displayed in the ward detail interface in FIG. 7 are different from those displayed in the ward detail interface in FIG. 6.

For example, referring to FIG. 6 and FIG. 7, the ward detail interfaces display the ward condition, risk management, nursing contents, inspection condition, catheter information, today's shift arrangement, today's/tomorrow's surgery, patient prompts, critical values, bed change information, frequent contacts, and the like.

For example, when the nurse performs a touch operation on the “material management” control in the home interface shown in FIG. 4, such as, by clicking the control with a finger, the electronic whiteboard device display a material management interface in response to the touch operation, and the material management interface displays drugs, consumables and quantity of devices in the current ward, such as, in a form of a list: “captopril, 12.5 mg*20/box, 10 boxes”, “disposable syringe, 20 ml, 20”.

For example, the memorandum may record any prompt contents for nurses, such as “nurse Zhang xx, from ward II of the internal medicine department, borrows five dexamethasone injections, Li XX, XXXX year, XX month, XX day, XX time).

For example, the overall nursing schedule of the wards includes the completion of infusion/treatment in the wards, such as Box “work schedule” in the home interface shown in FIG. 4.

For example, the patient prompts include prompts of attentions to patients on corresponding beds at proper time, such as “Bed No. 12, April 9, no food or water after 20:00, venous blood collection in the morning at April 10”.

In some possible implementations, the home interface also displays a shift-handover record create control.

In some possible implementations, the home display also displays a shift-handover record format set control.

The trigger and response manners of the shift-handover record create control and the shift-handover record format set control are described in S20 and S30, respectively.

S20: receiving, by the electronic whiteboard device, shift-handover permission application information and verifying a shift-handover permission; acquiring shift-handover information and generating a shift-handover record in response to an operation on the shift-handover record create control displayed in the home interface.

In this way, nurses to be relieved may submit electronic shift-handover records efficiently and conveniently by means of the electronic whiteboard device with an interaction device, and the electronic shift-handover records and information are standard, so that information omissions or errors are avoided. The nurses to be relieved may check the submitted shift-handover records conveniently at any time by means of the electronic whiteboard device. In this way, the shift-exchange convenience and efficiency are further improved for nurses, and omissions or errors of shift-exchange information are avoided.

In addition to the manner of generating the shift-handover record by the electronic whiteboard device in response to an operation performed by the nurse to be relieved, the shift-handover record may also be generated in such as manner that the nurse to be relieved logs in another terminal such as a computer device or a PDA (personal digital assistant), submit the shift-handover record by means of the terminal and send the shift-handover record to the electronic whiteboard device, that is, S20 may be replaced with: receiving, by another terminal, the shift-handover permission application information and verifying a shift-handover permission; acquiring shift-handover information and generating a shift-handover record in response to an operation on the displayed shift-handover record create control displayed, and sending the shift-handover record to the electronic whiteboard device.

In some feasible implementations, the step of receiving, by the electronic whiteboard device, the shift-handover permission application information and verifying the shift-handover permission comprises: receiving the shift-handover permission application information in NFC data and verifying the shift-handover permission.

In this way, the nurse to be relieved can conveniently log in the electronic whiteboard device and verify his/her permission by means of a work card with an RFID or NFC tag. It may be understood that, in this implementation, the electronic whiteboard device should be provided with an NFC module such as an RFID reader or an NFC reader.

In addition, similar to the step of receiving the display set permission application information and verifying the display set permission, in the step of receiving the shift-takeover permission application information and verifying the shift-takeover permission, the shift-takeover permission may be verified in other manners such as by displaying a login control and inputting account information, including a username and a password, input by means of the login control, or by face recognition, fingerprint recognition, iris recognition or voice recognition.

In some possible implementations, acquiring the shift-handover information comprises:

acquiring the shift-handover information from a database; and/or

displaying a third interface displaying an input control, and acquiring shift-handover information input by means of the input control.

In this way, a nurse to be relieved does not need to manually filling the shift-handover record with all information such as ward information and patent information, and all shift-turnover information, including the ward information and patent information, within the work time of the nurse to be relieved may be acquired from a database, in which data in information systems of different hospitals is stored, by means of the electronic whiteboard device to automatically generate a shift-turnover record; besides, prompt information such as remarks and attentions input by the nurse to be relieved, may be appended to make the shift-handover record more comprehensively. In this way, the shift-exchange convenience and efficiency are further improved for nurses, and omissions or errors of shift-exchange information are avoided.

S30: displaying, by the electronic whiteboard device, a shift-exchange interface, wherein the shift-exchange interface displays shift-turnover information corresponding to the shift-handover record, and a shift-takeover control.

Wherein, if the shift-handover record is generated by the electronic whiteboard device in S20, the electronic whiteboard device displays the shift-exchange interface after generating the shift-handover record; or, if the shift-handover record is generated by the nurse to be relieved by means of other terminals, the electronic whiteboard device receives the shift-handover record from other terminals and then displays the shift-exchange interface.

In some possible implementations, the shift-handover information corresponding to the shift-handover record includes general ward information and patient information corresponding to the current shift exchange, and the patient information includes basic information, illness information and nursing information of the patients.

Wherein, the basic information of the patients may include room number (the room numbers are ordered in all the rooms rather than in the wards), bed number, name and diagnosis number, the illness information of the patients includes diagnosis information, medical history information, surgical information and doctor's advices, and the nursing information includes nursing level, nursing remarks, and the like.

In some possible implementations, the shift-handover information corresponding to the shift-handover record further includes: general ward information corresponding to the previous shift exchange, and patient illness information, corresponding to the previous shift exchange, of patients whose patient illness information corresponding to the current shift exchange has changed with respect to the previous shift exchange.

In this way, the nurse on duty can gain a better understanding of illness changes of patients to better serve the patients.

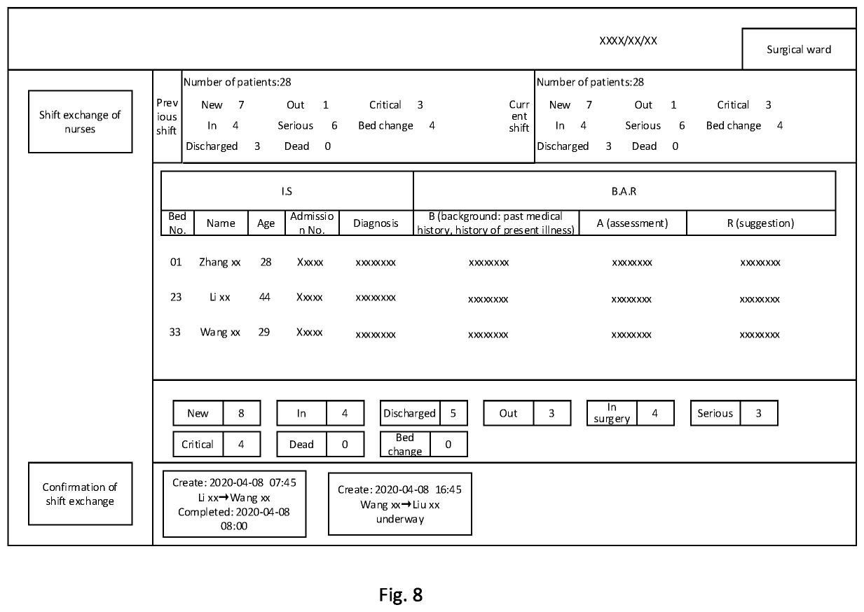

In one specific example, as shown in FIG. 8, the shift-turnover interface displays general ward information in the previous shift and ward generation information in the current shift, a shift-turnover confirm control (at the left bottom of FIG. 8), information of a nurse in the previous shift, the shift-exchange time, information of a nurse in the current shift, and patient information in the current shift, including bed number, name, age, admission number (diagnosis number), diagnosis results and nursing suggestions given to patients by the nurse to be relieved. Wherein, a patient control corresponding to the bed number or the patient name may be set, so that the electronic whiteboard device is able to display a patient detail interface, similar to the one shown in FIG. 5, when the nurse to be on duty performs a touch operation on the patient control, such as, by clicking the control with a finger, and thus, the nurse to be on duty may gain a more comprehensive understanding of the condition of the patients.

In some possible implementations, the interaction method provided by this embodiment further comprises:

receiving shift-handover record format set permission application information and verifying a shift-handover record format set permission; and

setting a format of the shift-handover record in response to an operation in the shift-handover record format set control displayed in the home interface.

The shift-handover record format set permission application information may be a first set permission application information.

Wherein, similar to the step of receiving the display set permission application information and verifying the display set permission, the step of receiving the shift-handover record format set permission application information and verifying the shift-handover record format set permission may be implemented by receiving the permission application information in NFC data and verifying the permission, and unnecessary details will no longer be given here.

In this way, personnel possessing a shift-handover record format set permission such as a ward manager and a charge nurse may modify the format of the shift-handover record conveniently, such as by adding or deleting information items, by means of the electronic whiteboard device, and thus, the interaction method provided by the present disclosure is able to meet different requirements of different wards (for example, wards of the inpatient departments of different hospitals may have different nursing requirements, and wards of the neurology department and surgery department of the same hospital may also have different nursing requirements).

In one specific example, considering that the elderly pays more attention to the catheter condition, catheter information may be added to the shift-handover record corresponding to a ward for the elderly, so the shift-turnover information corresponding to the shift-turnover record displayed in the shift-exchange interface may include the catheter type, critical level, catheterizing cycle and dressing time. It should be noted that if information to be added is standard and comprehensive, data stored in a database may be associated directly by system setting to obtain data columns, and a program code does not need to be modified.

It should be noted that the shift-handover record format set control is not only displayed in the home interface, and may also be displayed in other interfaces such as the shift-exchange interface. In addition, the display set control may also be displayed in other interfaces.

S40: receiving, by the electronic whiteboard device, shift-takeover permission application information and verifying a shift-takeover permission; and confirming that a shift exchange is completed in response to an operation on the shift-takeover confirm control.

The electronic whiteboard device maybe other electronic devices with interaction devices.

In some feasible implementations, the step of receiving the shift-takeover permission application information and verifying the shift-takeover permission comprises: receiving the shift-takeover permission application information in NFC data and verifying the shift-takeover permission.

Wherein, similar to the step of receiving the display set permission application information and verifying the display set permission, the step of receiving the shift-takeover permission application information and verifying the shift-takeover permission may be implemented by receiving the shift-takeover permission application information in NFC data and verifying the shift-takeover permission, and unnecessary details will no longer be given here.

In some feasible implementations, after it is confirmed that the shift exchange is completed, the interaction method provided by this embodiment further comprises: displaying the home interface; or, quitting the shift-exchange interface and returning to the home interface.

In one specific example, the following rule may be set: if the current shift handover is not confirmed, it is not allowed to create a new shift-handover record to avoid confusions. The rule may be set specifically as needed by the actual scene, for example, the shift-turnover rule for a whole group may be different from the shift-turnover rule for a single person. This embodiment has no limitation to this aspect.

In one specific example, after the shift exchange is completed, the charge nurse may fill in a shift-turnover summary based on the shift-turnover record by means of a summary control displayed in the home interface to summarize the completion and quality of the nursing work in the previous shift and assign key emphasis in work in this shift. The summary control may provide templates and common phrases to facilitate filling-in of the shift-turnover summary. In addition, after the shift exchange is completed, the nurse to be on duty may check the shift-exchange result and historical shift-exchange records based on a check control displayed in the home interface.

By adoption of the interaction method provided by this embodiment, nurses may exchange shifts electronically by means of the electronic whiteboard device installed at the nurse station to realize paperless office. Wherein, nurses to be relieved and nurses to be on duty, specifically the nurses to be on duty, can check shift-handover information at any time by means of the electronic whiteboard device to gain a comprehensive and detailed understanding of the ward condition and the patent condition, and do not need to carry a great number of paper shift-exchange records or manually go through the records to check the shift-turnover information including the ward information and patent information. After the permission of the nurses to be on relieved is verified as valid, the nurses may submit electronic shift-handover records efficiently and conveniently by means of the electronic whiteboard device, and the electronic shift-handover records and information are standard, so that information omissions or errors are avoided; and the nurses to be relieved may check the submitted shift-handover records conveniently at any time by means of the electronic whiteboard device. After the permission of the nurses to be on duty is verified as valid, the nurses can confirm the completion of the shift exchange directly by means of the electronic whiteboard device, that is, “one-click confirmation” is realized. So, the interaction method provided by this embodiment may greatly improve the shift-exchange convenience and efficiency for nurses.

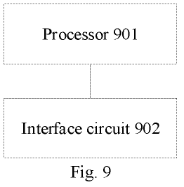

One embodiment of the present disclosure provides a chip system. As shown in FIG. 9, the chip system comprises at least one processor 901 and at least one interface circuit 902. The processor 901 and the interface circuit 902 may be interconnected by a circuit. For example, the interface circuit 902 may receive a signal from other devices (such as a memory of a mobile terminal). For another example, the interface circuit 902 may send a signal to other devices (such as the processor 901). Illustratively, the interface circuit 902 may read an instruction stored in the memory and send the instruction to the processor 901. When the instruction is executed by the processor 901, an electronic device performs the steps, executed by the electronic device 100 (such as the electronic whiteboard device) in the above embodiment. Of course, the chip system may also comprise other discrete components, and the embodiments of the present disclosure have not specific limitation in this aspect.

It may be understood that, to fulfill the above functions, the terminal comprises corresponding hard structures and/or software modules for executing these functions. Those skilled in the art would easily appreciate that the units and arithmetic steps in the examples described in conjunction with the embodiments of the present disclosure may be implemented by hardware or the combination of hardware and computer software. Whether a function is executed by hardware or computer software for driving the hardware depends on the specific application and design constraints of the technical solution. For each specific application, professionals may adopt different methods to fulfill the functions described, without departing from the scope of the embodiments of the present disclosure.

In the embodiments of the present disclosure, the terminal may be divided into different functional modules according to the examples of the method. For example, the terminal may be divided into different functional modules according to different functions; or, two or more functions may be integrated in one processing module that may be implemented in the form of hardware or in the form of a software functional module. It should be noted that the modules in the embodiments of the invention are illustrative ones based on logic functions, and other functional modules may be configured in actual implementation.

With reference to the description of the above implementations, those skilled in the art may clearly understand that, for the sake of a simple and brief description, the above functional modules are merely illustrative ones, and in actual application, the above functions may be fulfilled by different functional modules as needed, that is, the internal structure of the device may be divided into different functional modules to complete all or part of the functions described above.

The specific working process of the system, device and units described above may be understood with reference to the corresponding process in the embodiments of the method, and will no longer be detailed here.

The functional units in the embodiments of the present disclosure may be integrated in one processing unit or be physically independent, or two or more units may be integrated in one unit. The integrated unit may be implemented in the form of hardware or in the form of a software functional module.

When implemented in the form of software functional units and sold or used as independent products, the integrated units may be stored in a computer-accessible memory. On the basis of such a understanding, the technical solution of the invention, or parts, making a contribution to the prior art, of the technical solution, or all or part of the technical solution may be embodied in the form of a software product which is stored in a memory comprising a plurality of requests to enable a computer device (such as a personal computer, a server or a network device) to perform all or part of the steps of the method in the above embodiments. The storage medium may be any media capable of storing a program code, such as a flash memory, a mobile hard disk, a read only memory, a random access memory, a diskette and an optical disk.

Obviously, the aforesaid embodiments of the present disclosure are merely examples for clearly explaining the present disclosure, and are not intended to limit the implementations of the present disclosure. Those ordinarily skilled in the art may make different variations or transformations based on the above description, and it is impossible to enumerate all possible implementations of the invention. All obvious variations or transformations extended from the technical solutions of the present disclosure should also fall within the protection scope of the present disclosure.

1. An interaction method, being applied to an electronic device with an interaction device, and comprising:
receiving a shift-handover record, and displaying a first interface, wherein the first interface displays shift-handover information corresponding to the shift-handover record, and a shift-takeover confirm control, and the shift-handover record is a shift-handover record by medical staff;
receiving shift-takeover permission application information and verifying a shift-takeover permission; and
confirming that a shift exchange is completed in response to an operation on the shift-takeover confirm control.

2. The method according to claim 1, wherein before the step of receiving the shift-handover record, the method further comprises:
displaying a second interface, wherein the second interface displays a shift-handover record create control;
receiving a shift-handover permission application information and verifying a shift-handover permission; and
acquiring shift-handover information and generating the shift-handover record in response to an operation on the shift-handover record create control.

3. The method according to claim 2, wherein the step of acquiring the shift-handover information comprises at least one of:
acquiring the shift-handover information from a database; and
displaying a third interface that display an input control, and acquiring the shift-handover information input by means of the input control.

4. The method according to claim 2, wherein:
the step of the receiving shift-handover permission application information and verifying the shift-handover permission comprises: receiving shift-handover permission application information in NFC data and verifying the shift-handover permission.

5. The method according to claim 2, wherein:
the step of receiving the shift-takeover permission application information and verifying the shift-takeover permission: receiving shift-takeover permission application information in NFC data and verifying the shift-takeover permission.

6. The method according to claim 1, wherein the shift-turnover information corresponding to the shift-turnover record includes general ward information and patient information corresponding to a current shift exchange, and the patient information includes basic information, illness condition and nursing information of patents.

7. The method according to claim 6, wherein the shift-handover information corresponding to the shift-handover records further includes: general ward information in a previous shift exchange, and patient illness information, corresponding to the previous shift exchange, of patients whose patient illness information corresponding to the current shift exchange has changed with respect to the previous shift exchange.

8. The method according to claim 2, wherein the second interface also displays a shift-handover record format set control, and the method further comprises:
receiving a first set permission application information and verifying a first set permission; and
setting a format of the shift-turnover record in response to an operation on the shift-handover record format set control.

9. The method according to claim 2, wherein the second interface also displays patient controls corresponding to patients in wards, and the method further comprises:
displaying a fourth interface in response to an operation on the patient controls, wherein the fourth interface displays corresponding patient information, and the patient information includes basic information, illness information and nursing information of patients.

10. The method according to claim 9, wherein the second interface also displays a display set control, and the method further comprises:
receiving second set permission application information and verifying a second set permission; and
setting information displayed by the second interface, and contents and display parameters of the controls in response to an operation on the display set control.

11. The method according to claim 2, wherein after the step of confirming that the shift exchange is completed, the method further comprises: displaying the second interface.

12. An electronic device, comprising a processor, a memory and an interaction device, wherein the memory and the interaction device are coupled to the processor, the memory is used to store a computer program code which includes a computer instruction, and when the processor reads the computer instruction from the memory, the electronic device is enabled to implement the interaction method according to claim 1.

13. The electronic device according to claim 12, wherein the electronic device is an electronic whiteboard device.

14. The electronic device according to claim 13, wherein the electronic device is installed in a medical office area.

15. A computer readable storage medium, comprising a computer instruction which enables, when run on a terminal, the terminal to implement the interaction method according to claim 1.

16. A computer program product comprising a computer readable code, when the computer readable code is implemented in a computing processing device, the computing processing device is made to execute the interaction method according to claim 1.

17. An electronic device, wherein the electronic device comprises:
a memory configured for storing a computer instruction executable by the processor; and
one or more processors, wherein the processors are configured for executing the computer instruction, to implement the interaction method according to claim 1.

18. The electronic device according to claim 17, wherein before the operation of receiving the shift-handover record, the processors further execute the operations comprising:
displaying a second interface, wherein the second interface displays a shift-handover record create control;
receiving a shift-handover permission application information and verifying a shift-handover permission; and
acquiring shift-handover information and generating the shift-handover record in response to an operation on the shift-handover record create control.

19. The electronic device according to claim 18, wherein the operation of acquiring the shift-handover information comprises at least one of:
acquiring the shift-handover information from a database; and
displaying a third interface that display an input control, and acquiring the shift-handover information input by means of the input control.

20. The electronic device according to claim 18, wherein:
the operation of the receiving shift-handover permission application information and verifying the shift-handover permission comprises:
receiving shift-handover permission application information in NFC data and verifying the shift-handover permission.
